(19) United States
(12) Patent Application Publication
(10) Pub. No.: US 2014/0073920 A1
(43) Pub. Date: Mar. 13, 2014

(54) ULTRASOUND DIAGNOSTIC APPARATUS AND ULTRASOUND IMAGE PRODUCING METHOD

(71) Applicant: FUJIFILM Corporation, Tokyo (JP)

(72) Inventor: Masafumi Noguchi, Tokyo (JP)

(73) Assignee: FUJIFILM Corporation, Tokyo (JP)

(21) Appl. No.: 14/023,318

(22) Filed: Sep. 10, 2013

(30) Foreign Application Priority Data
Sep. 13, 2012 (JP) 2012-201361

(51) Int. Cl. A61B 8/08

(52) U.S. Cl. CPC A61B 8/5215
USPC 600/437

(57) ABSTRACT

The ultrasound diagnostic apparatus includes an image data generator for generating ultrasound image data based on reception data, a resolution adjuster for adjusting a resolution of the ultrasound image data to generate adjusted ultrasound image data, an edge determiner for determining an edge of a structure contained in an ultrasound image based on the adjusted ultrasound image data to obtain edge information of the structure, and an image processor for performing image processing including denoising or edge enhancement on the adjusted ultrasound image data or on the ultrasound image data generated in the image data generator based on the edge information to generate processed ultrasound image data.

### BACKGROUND OF THE INVENTION

The present invention relates to an ultrasound diagnostic apparatus and an ultrasound image producing method, and in particular, an ultrasound diagnostic apparatus which transmits and receives ultrasound from an ultrasound probe, produces an ultrasound image based on the reception signal, and displays the ultrasound image on a display.

Conventionally, ultrasound diagnostic apparatus using ultrasound images are employed in medicine. Generally, in this type of ultrasound diagnostic apparatus, a transducer array provided in an ultrasound probe transmits an ultrasonic beam toward the inside of a subject's body, the ultrasonic echo from the subject is received by the transducer array, and the resulting reception signals are electrically processed by a diagnostic apparatus body to produce an ultrasound image.

In recent years, as disclosed in, for example, JP 4789854 B and JP 2012-50816 A, an ultrasound diagnostic apparatus which produces an ultrasound image with the improved image quality by performing multiresolution analysis on the ultrasound image and thereafter performing image processing such as denoising and edge enhancement when producing the ultrasound image has been proposed.

### SUMMARY OF THE INVENTION

When it is desired to clearly observe a particular structure in an ultrasound image in the ultrasound diagnosis, by employing the multiresolution decomposition disclosed by JP 478954 B and JP 2012-50816 A, a plurality of resolutions can be selected based on the result of multiresolution decomposition in accordance with the size of the structure in the ultrasound image, allowing the structure to be observed. Multiresolution decomposition, however, changes the resolution of an ultrasound image in a phased manner at a given rate such as by ½. Hence, there was a problem that when the frequency range suitable for extracting the interested structure within an ultrasound image lies in between two successive multiple-resolution images resulting from multiresolution decomposition, the interested structure cannot be properly extracted even if using any multiple-resolution image resulting from multiresolution decomposition.

The present invention has an object to provide an ultrasound diagnostic apparatus and an ultrasound image producing method which can produce an ultrasound image allowing easy observation of an interested structure such as a superficial tissue, blood vessel and diaphragm contained in an ultrasound image in the ultrasound diagnosis.

In order to attain the object described above, the present invention provides an ultrasound diagnostic apparatus which transmits ultrasound from an ultrasound probe toward a subject and produces an ultrasound image in a diagnostic apparatus body based on reception data obtained, comprising an image data generator for generating ultrasound image data based on the reception data, a resolution adjuster for adjusting a resolution of the ultrasound image data generated in the image data generator to generate adjusted ultrasound image data, an edge determiner for determining an edge of a structure contained in an ultrasound image based on the adjusted ultrasound image data generated in the resolution adjuster to obtain edge information of the structure, and an image processor for performing image processing including denoising or edge enhancement on the adjusted ultrasound image data generated in the resolution adjuster or on the ultrasound image data generated in the image data generator based on the edge information obtained in the edge determiner to generate processed ultrasound image data.

It is preferable that the ultrasound diagnostic apparatus further comprises a decimation section for performing decimation on the ultrasound image data to generate downsampled ultrasound image data, and the resolution adjuster preferably adjusts a resolution of the downsampled ultrasound image data generated in the decimation section to generate adjusted ultrasound image data.

The resolution adjuster preferably adjusts the resolution of the ultrasound image data in accordance with a central frequency of the ultrasound probe, a bandwidth of the transmitted ultrasound, a density of scanning lines of the ultrasound probe, filter characteristics of a detection filter for wave detection processing on the reception data, or an imaging technique such as harmonic imaging, to thereby generate the adjusted ultrasound image data, and the resolution adjuster preferably adjusts the resolution of the ultrasound image data in proportion to a central frequency of the ultrasound probe to generate the adjusted ultrasound image data.

It is preferable that the ultrasound diagnostic apparatus further comprises a scan converter for performing coordinate conversion on the ultrasound image data generated in the image data generator for display, and a resolution restoring section for restoring a resolution of the adjusted ultrasound image data subjected to the image processing in the image processor back to the resolution before adjustment.

It is preferable that the ultrasound diagnostic apparatus further comprises a multiresolution decomposition section for performing multiresolution decomposition on the adjusted ultrasound image data generated in the resolution adjuster to decompose the adjusted ultrasound image data into a plurality of decomposed ultrasound image data having different resolutions, and a multiresolution recomposition section for recomposing the plurality of decomposed ultrasound image data having different resolutions, wherein the edge determiner determines the edge of the structure contained in the ultrasound image based on the plurality of decomposed ultrasound image data having the different resolutions resulting from decomposition in the multiresolution decomposition section, to thereby obtain the edge information of the structure, and the image processor performs the image processing on each of the plurality of decomposed ultrasound image data based on the corresponding edge information obtained in the edge determiner to generate a plurality of processed decomposed ultrasound image data having the different resolutions and outputs the plurality of processed decomposed ultrasound image data to the multiresolution recomposition section.

It is preferable that the ultrasound diagnostic apparatus further comprises a multiresolution decomposer for performing multiresolution decomposition on the ultrasound image data generated in the image data generator to decompose the ultrasound image data into a plurality of decomposed ultrasound image data having different resolutions, a resolution restoring section for restoring a resolution of each of the plurality of decomposed ultrasound image data having the different resolutions to generate a plurality of restored decomposed ultrasound image data, and a multiresolution recomposition section for recomposing the plurality of restored decomposed image data generated in the resolution restoring section, wherein the resolution adjuster adjusts a resolution of each of the plurality of decomposed ultrasound image data having the different resolutions resulting from multiresolution decomposition in the multiresolution decomposition section to generate a plurality of adjusted decomposed ultrasound image data, the edge determiner determines the edge of the structure contained in the ultrasound image based on the plurality of adjusted decomposed ultrasound image data generated in the resolution adjuster, to thereby obtain the edge information of the structure, and the image processor performs the image processing on each of the plurality of adjusted decomposed ultrasound image data based on the corresponding edge information to generate a plurality of processed decomposed ultrasound image data having the different resolutions and outputs the plurality of processed decomposed ultrasound image data to the resolution restoring section.

The ultrasound diagnostic apparatus may further comprise a first resolution restoring section for restoring a resolution of the adjusted ultrasound image data generated in the resolution adjuster to generate first restored data, a subtracter for subtracting the first restored data from the ultrasound image data to generate subtracted data, a second resolution restoring section for restoring a resolution of the processed ultrasound image data generated in the image processor to generate second restored data, and an adder for adding the subtracted data and the second restored data to generate added data.

In addition, the present invention provides a method for producing an ultrasound image in which ultrasound is transmitted from an ultrasound probe toward a subject and the ultrasound image is generated based on reception data obtained, comprising the steps of generating ultrasound image data based on the reception data, adjusting a resolution of the generated ultrasound image data to generate adjusted ultrasound image data, determining an edge of a structure contained in the ultrasound image based on the adjusted ultrasound image data thus generated to obtain edge information of the structure, and performing image processing including denoising or edge enhancement on the adjusted ultrasound image data or the ultrasound image data based on the edge information thus obtained to generate processed ultrasound image data.

According to the present invention, in the ultrasound diagnosis, an ultrasound image which allows easy observation of an interested structure can be produced by adjusting the resolution of the ultrasound image in accordance with the interested structure in the ultrasound image, detecting an edge of the structure based on the ultrasound image after resolution adjustment, and performing appropriate image processing.

### BRIEF DESCRIPTION OF THE DRAWINGS

FIG. 1 is a block diagram illustrating a whole configuration of the ultrasound diagnostic apparatus according to an embodiment 1 of the invention.

FIG. 2 is a block diagram illustrating a detailed configuration of an image producer in the ultrasound diagnostic apparatus according to the embodiment 1 of the invention.

FIG. 3 is a block diagram illustrating a detailed configuration of an image producer in the ultrasound diagnostic apparatus according to an embodiment 2 of the invention.

FIG. 4 is a block diagram illustrating a detailed configuration of an image producer in the ultrasound diagnostic apparatus according to an embodiment 3 of the invention.

FIG. 5 is a block diagram illustrating a detailed configuration of an image producer in the ultrasound diagnostic apparatus according to a modification example of the embodiment 3 of the invention.

FIG. 6 is a block diagram illustrating a detailed configuration of an image producer in the ultrasound diagnostic apparatus according to an embodiment 4 of the invention.

FIG. 7 is a block diagram illustrating a detailed configuration of an image producer in the ultrasound diagnostic apparatus according to an embodiment 5 of the invention.

FIG. 8 is a block diagram illustrating a detailed configuration of an image producer in the ultrasound diagnostic apparatus according to an embodiment 6 of the invention.

FIG. 9 is a block diagram illustrating a detailed configuration of an image producer in the ultrasound diagnostic apparatus according to an embodiment 7 of the invention.

### DETAILED DESCRIPTION OF THE INVENTION

Embodiments of the present invention will now be described below based on the appended drawings.

### Embodiment 1

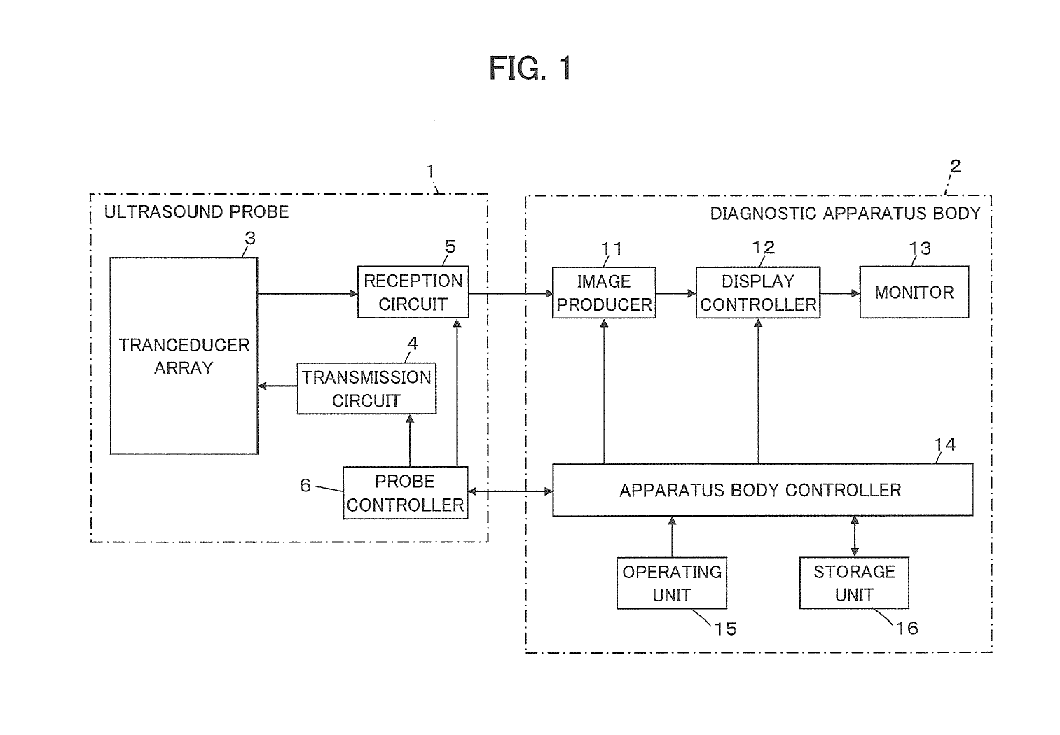

FIG. 1 is a block diagram illustrating a whole configuration of the ultrasound diagnostic apparatus according to the embodiment 1. The ultrasound diagnostic apparatus comprises an ultrasound probe 1 and a diagnostic apparatus body 2 connected to the ultrasound probe 1.

The ultrasound probe 1 has a transducer array 3, and the transducer array 3 is connected to a transmission circuit 4 and a reception circuit 5, to both of which a probe controller 6 is connected.

The diagnostic apparatus body 2 comprises an image producer 11 connected to the reception circuit 5 of the ultrasound probe 1. The image producer 11 is connected in sequence to a display controller 12 and a monitor 13. An apparatus body controller 14 is connected to the image producer 11 and the display controller 12.

The apparatus body controller 14 is further connected to an operating unit 15 and a storage unit 16.

The probe controller 6 of the ultrasound probe 1 and the apparatus body controller 14 of the diagnostic apparatus body 2 are connected to each other.

The transducer array 3 of the ultrasound probe 1 includes a plurality of ultrasound transducers arrayed in one dimension or two dimensions. These ultrasound transducers each transmit ultrasonic waves according to transmission signals supplied from the transmission circuit 4 and receive ultrasonic echoes from the subject to output reception signals. Each of the ultrasound transducers comprises a vibrator composed of a piezoelectric body and electrodes provided on both ends of the piezoelectric body. The piezoelectric body is composed of, for example, a piezoelectric ceramic represented by a PZT (lead zirconate titanate), a piezoelectric polymer represented by PVDF (polyvinylidene fluoride), or a piezoelectric single crystal represented by PMN-PT (lead magnesium niobate-lead titanate solid solution).

When a pulsed voltage or a continuous-wave transmission signal voltage is applied to the electrodes of such a vibrator, the piezoelectric body expands and contracts to cause the vibrator to generate pulsed or continuous ultrasonic waves. These ultrasonic waves are synthesized to form an ultrasonic beam. Upon reception of propagating ultrasonic waves, each vibrator expands and contracts to produce an electric signal, which is then outputted as a reception signal of the ultrasonic waves.

The transmission circuit 4 includes, for example, a plurality of transmitters and adjusts the delay amounts for transmission signals based on a transmission delay pattern selected according to a control signal transmitted from the probe controller 6 so that the ultrasonic waves transmitted from the plurality of ultrasound transducers of the transducer array 3 form an ultrasonic beam, and supplies the ultrasound transducers with delay-adjusted transmission signals.

The reception circuit 5 amplifies and A/D-converts the reception signals transmitted from the ultrasound transducers of the transducer array 3, and then performs reception focusing processing by providing the reception signals with respective delays according to the sonic speed or sonic speed distribution that is set based on a reception delay pattern selected according to the control signal transmitted from the probe controller 6 and adding them up. This reception focusing processing yields reception data (sound ray signals) having the ultrasonic echoes well focused.

The probe controller 6 controls the respective components of the ultrasound probe 1 according to various control signals transmitted from the apparatus body controller 14 of the diagnostic apparatus body 2. The reception data generated by the reception circuit 5 according to the instruction of the probe controller 6 is sequentially outputted to the image producer 11 of the diagnostic apparatus body 2.

Meanwhile, the image producer 11 of the diagnostic apparatus body 2 receives reception data generated by the reception circuit 5 of the ultrasound probe 1 to produce ultrasound image data, converts the ultrasound image data to display image data which can be displayed on the monitor 13 and outputs the display image data to the display controller 12.

The display controller 12 causes the monitor 13 to display an ultrasound image based on the display image data inputted from the image producer 11.

The monitor 13 includes a display device such as an LCD, for example, and displays an ultrasound image under the control by the display controller 12.

The operating unit 15 includes various operation buttons for input operations by an operator. The operator can input a measurement depth and a measurement mode for the diagnosis through the operating unit 15.

The storage unit 16 stores, for example, an operation program and may be constituted by, for example, a recording medium such as a hard disk, a flexible disk, an MO, an MT, a RAM, a CD-ROM, a DVD-ROM, an SD card, a CF card, or a USB memory, or a server.

The apparatus body controller 14 controls the components in the diagnostic apparatus body 2 according to various instruction signals and the like entered by the operator using the operating unit 15.

The image producer 11 and the display controller 12 are each constituted by a CPU and an operation program for causing the CPU to perform various kinds of processing, but they may be each constituted by a digital circuit.

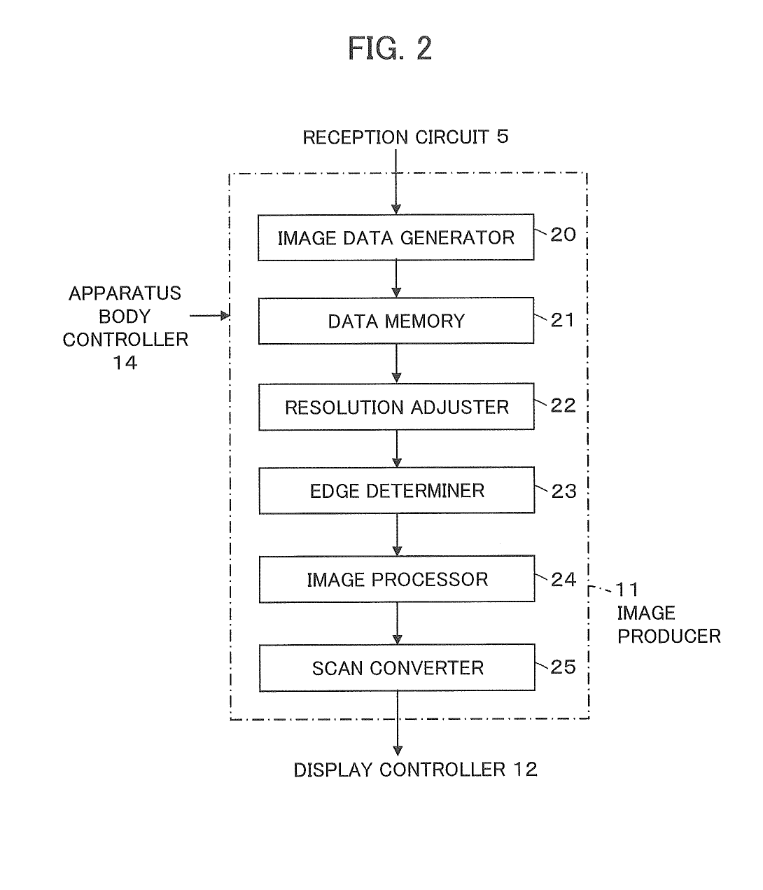

Next, a detailed configuration of the image producer 11 of the diagnostic apparatus body 2 is illustrated in the block diagram of FIG. 2.

The image producer 11 includes an image data generator 20 connected to the reception circuit 5 of the ultrasound probe 1, and in addition, a data memory 21, a resolution adjuster 22, an edge determiner 23, an image processor 24 and a scan converter 25 which are connected in sequence from the image data generator 20 onward. Then, the scan converter 25 is connected to the display controller 12.

The image data generator 20 corrects attenuation in the reception data generated by the reception circuit 5 of the ultrasound probe 1 according to the distance, i.e., the depth at which the ultrasonic waves are reflected, and then performs envelope detection processing to generate B mode image data, which is tomographic image information on a tissue inside the subject's body. The ultrasound image data generated in the image data generator 20 is outputted to the data memory 21.

The data memory 21 sequentially stores the ultrasound image data which is sequentially generated in the image data generator 20 and outputs the stored ultrasound image data to the resolution adjuster 22 according to the instruction from the apparatus body controller 14.

The resolution adjuster 22 adjusts the resolution of the ultrasound image data to allow easy edge detection of an interested structure in the ultrasound image based on the ultrasound image data and generates the adjusted ultrasound image data. In particular, the resolution of the ultrasound image data is adjusted through processing of enlarging or reducing the ultrasound image based on the ultrasound image data. Examples of the interested structure for observation in the ultrasound diagnosis include a superficial tissue, blood vessel and diaphragm contained in the ultrasound image for observation in the ultrasound diagnosis.

When the resolution of the ultrasound image data is adjusted according to the central frequency of (the transducer array 3 of) the ultrasound probe 1, an edge of the interested structure in the ultrasound image based on the adjusted ultrasound image data can be readily detected. In particular, the resolution of the ultrasound image data is preferably adjusted so as to be proportional to the central frequency of the ultrasound probe 1. This is because, generally, the resolution capability of the resulting ultrasound image is enhanced in proportion to the central frequency, so the smaller structure can be sharply drawn by raising the central frequency.

Accordingly, the resolution adjuster 22 adjusts the resolution of the ultrasound image data according to the central frequency of the ultrasound probe 1, for example.

When the ultrasound probe 1 used is a convex probe having a central frequency of about 3 to 4 MHz, a resolution of, for example, 0.4 mm/pixel in the depth direction and 0.5 deg/pixel in the scanning line direction is suitable to extract a structure such as a superficial tissue, blood vessel and diaphragm in the ultrasound image. When the ultrasound probe 1 used is a linear probe having a central frequency of about 7 to 8 MHz, a resolution is preferably set to about twice the resolution of the above-described convex probe, that is, 0.2 mm/pixel in the depth direction and 0.25 deg/pixel in the scanning line direction.

Since a size of the speckle pattern which is normally deemed as a noise also depends on the central frequency of the ultrasound probe 1, it is preferable that the resolution be appropriately adjusted to depend on the central frequency of the ultrasound probe 1, in order to effectively remove the speckle noise.

The resolution capability and a size of the speckle pattern of an image vary depending on a frequency and a bandwidth of the ultrasonic waves transmitted from the ultrasound probe 1, a density of the scanning lines of the ultrasound probe 1, filter characteristics of a detection filter for wave detection processing on the reception data in the image data generator 20, and an imaging technique such as the harmonic imaging. Hence, the resolution may be adjusted in accordance with them.

The edge determiner 23 detects an edge of the structure from the ultrasound image based on the adjusted ultrasound image data, the ultrasound image corresponding to the adjusted ultrasound image data whose resolution is adjusted in the resolution adjuster 22, and obtains the edge information of the structure. The thus-obtained edge information of the structure is outputted to the image processor 24 together with the adjusted ultrasound image data. The edge information of the structure includes the frequency component information in the ultrasound image data.

Based on the edge information of the structure in the adjusted ultrasound image obtained in the edge determiner 23, the image processor 24 performs image processing including denoising or edge enhancement on the adjusted ultrasound image data to generate the processed ultrasound image data. For example, the adjusted ultrasound image data is subjected to denoising by applying a given low-pass filter in accordance with the edge information of the structure and to edge enhancement of the structure by amplifying a given band component based on the edge information of the structure. The processed ultrasound image data generated in the image processor 24 is outputted to the scan converter 25.

In order for the monitor 13 to display the processed ultrasound image data generated in the image processor 24, the scan converter 25 performs display processing such as raster conversion and gradation processing thereon to generate the display image data and outputs the display image data to the display controller 12.

Next, an operation of the ultrasound diagnostic apparatus according to the embodiment 1 of the invention will be described.

The power switch of the diagnostic apparatus body 2 is turned on to supply power to the respective components inside the diagnosis apparatus body 2 and the ultrasound probe 1, thereby starting up the ultrasound diagnostic apparatus.

An operator brings the ultrasound probe 1 in contact with a diagnosis site on the subject to start the ultrasound diagnosis.

Ultrasonic beams are sequentially transmitted to a subject from the plurality of ultrasound transducers of the transducer array 3 in accordance with the drive signal from the transmission circuit 4 of the ultrasound probe 1, and the reception signals from the subject which are received by the plurality of ultrasound transducers are sequentially outputted to the reception circuit 5, to generate the reception data. The reception data is sequentially outputted to the image producer 11 in the diagnosis apparatus body 2.

The image data generator 20 in the image producer 11 sequentially generates the ultrasound image data based on the reception data thus received and sequentially outputs the ultrasound image data to the data memory 21. The data memory 21 sequentially stores the ultrasound image data and, in addition, outputs the ultrasound image data stored in the data memory 12 to the resolution adjuster 22 according to the instruction from the apparatus body controller 14. In an example thereof, ultrasound image data for one frame is outputted to the resolution adjuster 22.

According to the central frequency of the ultrasound probe 1, the resolution adjuster 22 performs enlargement/reduction processing on the ultrasound image based on the ultrasound image data and generates the adjusted ultrasound image data whose resolution has been adjusted. The adjusted ultrasound image data thus generated is outputted to the edge determiner 23.

The edge determiner 23 detects an edge of the structure in the ultrasound image based on the adjusted ultrasound image data and obtains the edge information of the structure. The thus-obtained edge information of the structure is outputted to the image processor 24 together with the adjusted ultrasound image data.

Based on the edge information of the structure obtained in the edge determiner 23, the image processor 24 performs the image processing including denoising or edge enhancement on the adjusted ultrasound image data. In denoising, for example, a low-pass filter to cut off high frequency components is applied to the adjusted ultrasound image data so as to reduce the high frequency components in the ultrasound image, while in edge enhancement, for example, a band-pass filter or a high-pass filter corresponding to the edge information of the structure is applied to the adjusted ultrasound image data so as to enhance the components in the frequency band in which an edge is included. Thereafter, a given coefficient is applied, and the resultant is added to the adjusted ultrasound image data which is input data to the image processor 24. Accordingly, the processed ultrasound image data thus processed in the image processor 24 is outputted to the scan converter 25.

The scan converter 25 performs the display processing such as raster conversion and gradation processing on the processed ultrasound image data to produce the display image data capable of being displayed on the monitor 13 and outputs the display image data to the display controller 12.

According to the instruction from the apparatus body controller 14, the display controller 12 causes the monitor 13 to display the ultrasound image based on the display image data, the ultrasound image having undergone denoising or edge enhancement and allowing easy observation of the interested structure.

The ultrasound diagnostic apparatus according to the embodiment 1 can obtain an ultrasound image allowing easy observation where the interested structure in the ultrasound image has been suitably enhanced for observation in the ultrasound diagnosis by appropriately reducing noises in the ultrasound image to suitably adjust the resolution of the ultrasound image data in the resolution adjuster 22, prior to image processing such as denoising and edge enhancement.

### Embodiment 2

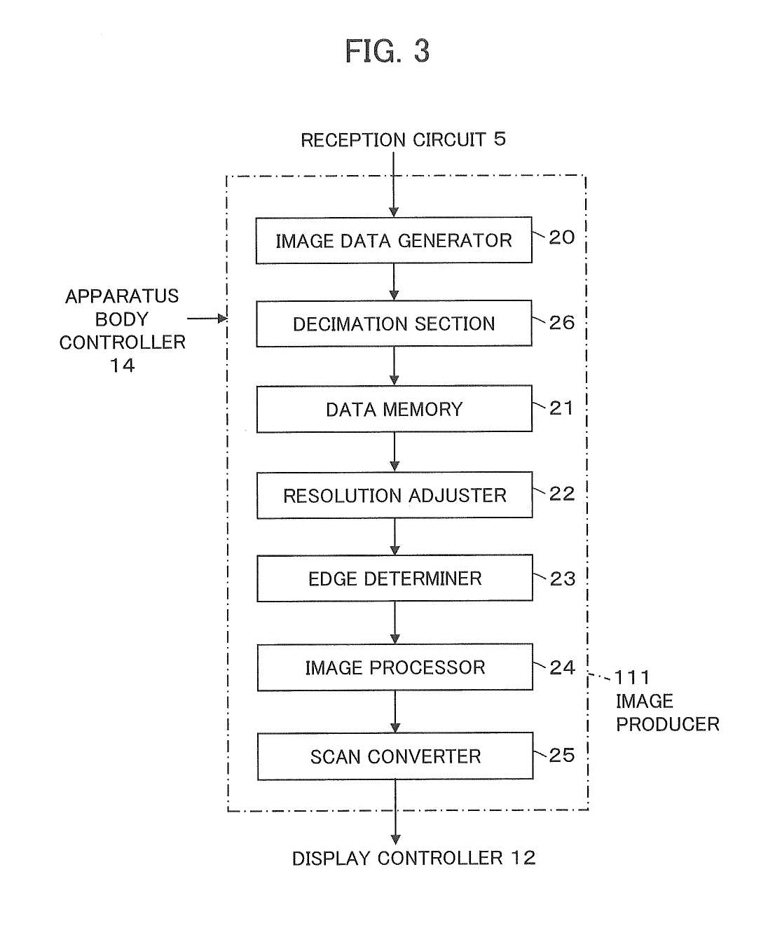

In the embodiment 1 described above, the image data generator 20 of the image producer 11 sequentially generates ultrasound image data, and the thus generated ultrasound image data is straightly stored in the data memory 21 in sequence. However, the ultrasound image data can be subjected to decimation (downsampling) before storage in the data memory 21, depending on the storage capacity of the data memory 21, the data volume of the ultrasound image data (which varies according to the scanning depth, the video replay time, etc.), the type of image processing performed on the ultrasound image data, the resolution of the monitor 13, etc. In an example, as illustrated in FIG. 3, a decimation section 26 is provided between an image data generator 20 and a data memory 21 in an image producer 111 so that the ultrasound image data sequentially generated in the image data generator 20 is subjected to decimation before being stored in the data memory 21, thereby sequentially generating downsampled ultrasound image data. The data memory 21 sequentially stores the downsampled ultrasound image data which is sequentially generated in the decimation section 26 and outputs the downsampled ultrasound image data for one frame to a resolution adjuster 22 according to the instruction from the apparatus body controller 14. The resolution adjuster 22 adjusts the resolution of the downsampled ultrasound image data in place of the ultrasound image data in the embodiment 1 to generate the adjusted ultrasound image data and outputs the adjusted ultrasound image data to an edge determiner 23. Similarly to the embodiment 1, the edge determiner 23 detects edge information of the structure and outputs the edge information together with the adjusted ultrasound image data to an image processor 24. The image processor 24 performs image processing on the adjusted ultrasound image data based on the edge information of the structure to generate the processed ultrasound image data and outputs the processed ultrasound image data to a scan converter 25.

In the embodiment 2, since the ultrasound image data is subjected to decimation before being stored in the data memory 21, the data memory 21 needs less capacity compared to the embodiment 1. In addition, a volume of each ultrasound image data becomes small so that the loads including those on the resolution adjuster 22 to adjust the resolution, the edge determiner 23 to detect the edges of the structure, and the image processor 24 to perform image processing can be reduced.

### Embodiment 3

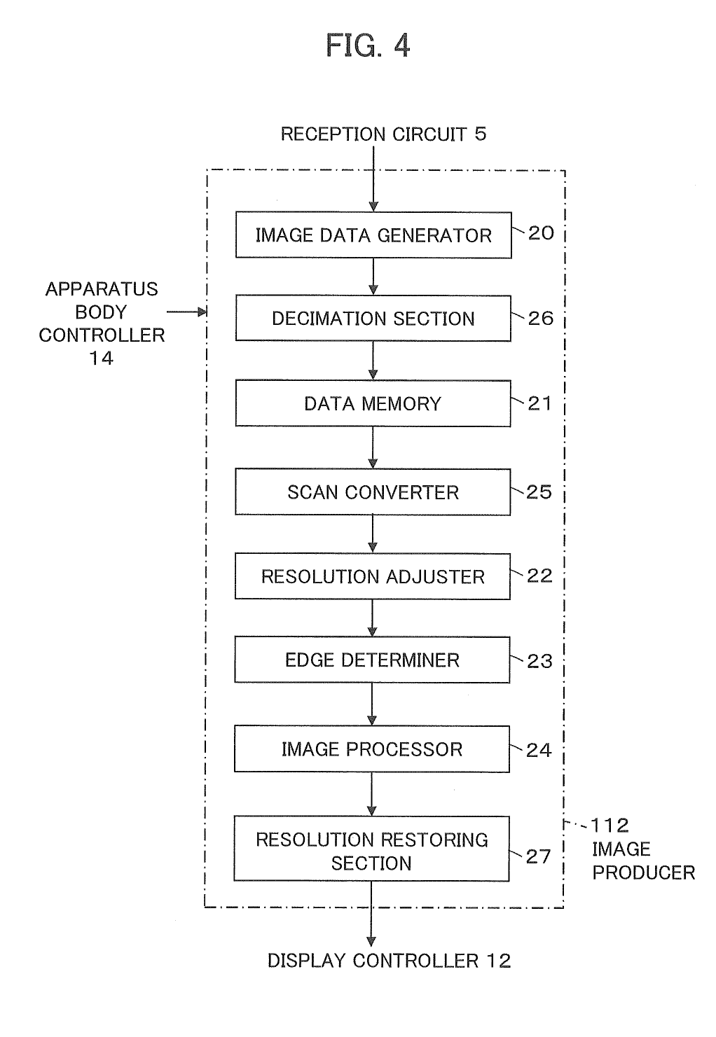

In the above-described embodiment 2, the scan converter 25 is provided between the image processor 24 and the display controller 12, and the display processing including raster conversion and gradation processing is performed according to the monitor 13 at the end of the series of image processing in the image producer 111. However, such the display processing can be performed before the resolution adjuster 22 adjusts the resolution. In an example, as illustrated in FIG. 4, a scan converter 25 is provided between a data memory 21 and a resolution adjuster 22 in an image producer 112, and a resolution restoring section 27 is provided between an image processor 24 and the display controller 12.

The scan converter 25 performs the display processing including raster conversion and gradation processing on the downsampled ultrasound image data for one frame which is outputted from the data memory 21 in accordance with the instruction of the apparatus body controller 14, generates the display image data which can be displayed on the monitor 13 and outputs the display image data to the resolution adjuster 22. The resolution adjuster 22 adjusts the resolution of the display image data generated in the scan converter 25 to generate the adjusted ultrasound image data and outputs the adjusted ultrasound image data to an edge determiner 23. The edge determiner 23 and an image processor 24 perform the same processing as in the embodiment 1 and embodiment 2 described above. The image processor 24 generates the processed ultrasound image data. However, the processed ultrasound image data has a resolution which has been adjusted in the resolution adjuster 22 so as to be changed from the resolution compatible with the monitor 13 and thus cannot be straightly displayed on the monitor 13. Accordingly, the processed ultrasound image data generated in the image processor 24 is outputted to the resolution restoring section 27, where the resolution of the processed ultrasound image data is restored so as to be equal to the resolution of the display image data before resolution adjustment in the resolution adjuster 22, and the restored image data is outputted to the display controller 12.

In the case of embodiment 1, also, the same effect as in the embodiment 3 can be obtained by providing the scan converter 25 between the data memory 21 and the resolution adjuster 22 and by providing a resolution restoring section 27 between the image processor 24 and the display controller 12.

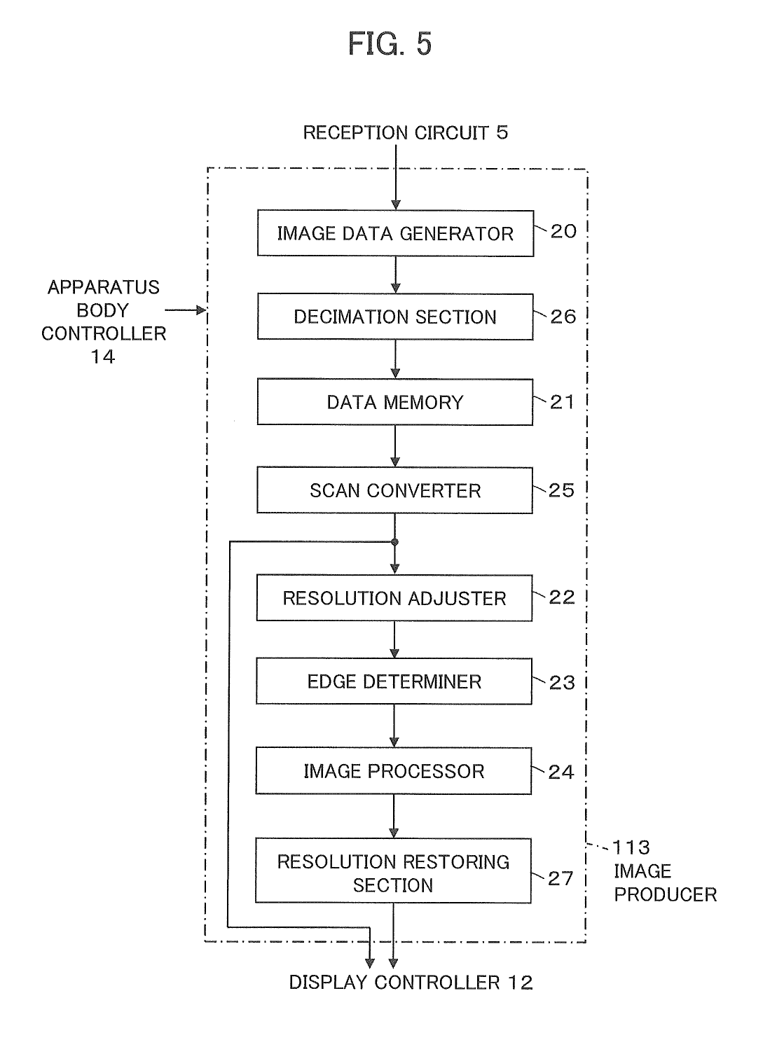

In addition, as a modification example of the embodiment 3, a scan converter 25 in an image producer 113 may be connected with a resolution adjuster 22 and, at the same time, directly connected with the display controller 12 as illustrated in FIG. 5.

The display image data is outputted from the scan converter 25 to the display controller 12 together with the above-described restored image data restored in a resolution restoring section 27.

The display controller 12 may cause the monitor 13 to display the ultrasound image which was subjected to image processing in the image processor 24 according to the instruction of the apparatus body controller 14 or to display the ultrasound image which was not subjected to image processing but directly outputted from the scan converter 25. Or, alternatively, both the ultrasound image subjected to image processing and the ultrasound image not subjected to image processing may be displayed at a time.

In the embodiment 3, as in the embodiment 1 and the embodiment 2 described above, it is possible to obtain an ultrasound image allowing easy observation where the interested structure in the ultrasound image has been suitably enhanced for observation in the ultrasound diagnosis by appropriately reducing noises in the ultrasound image to suitably adjust the resolution of the ultrasound image data in the resolution adjuster 22 prior to image processing such as denoising and edge enhancement.

In addition, in the modification example of the embodiment 3, by displaying the ultrasound image which was subjected to the given image processing and the ultrasound image which was not subjected to image processing together on the monitor 13 as described above, for example, both the ultrasound images can be directly compared with each other.

### Embodiment 4

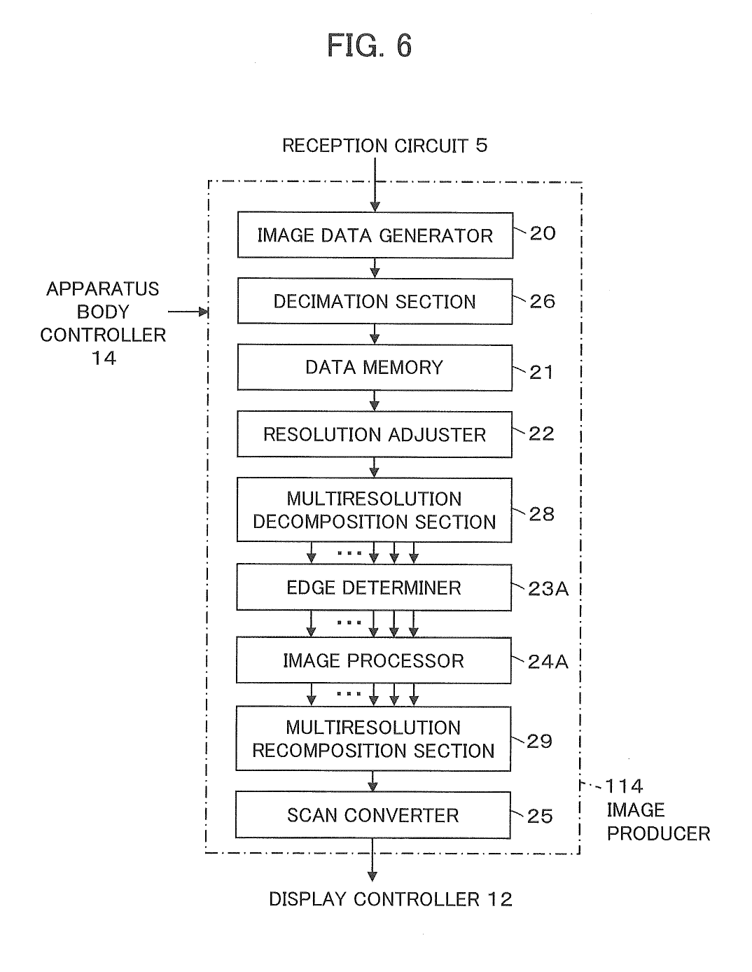

In the ultrasound diagnosis, when an ultrasound image contains a plurality of interested structures, and suitable resolutions for detection of their edges are different among the structures, an ultrasound image in which each of the plurality of structures is independently enhanced cannot be obtained in the above-described embodiment 2. However, through the use of multiresolution decomposition, a plurality of decomposed ultrasound image data having different resolutions are generated, and each of the plurality of decomposed ultrasound image data is subjected to image processing such as edge detection, denoising and edge enhancement, thereby enabling to obtain an ultrasound image in which each of the plurality of structures is independently enhanced. In an example, as illustrated in FIG. 6, an image producer 114 includes an image data generator 20 connected to the reception circuit 5 of the ultrasound probe 1, and in addition, a decimation section 26, a data memory 21, a resolution adjuster 22, a multiresolution decomposition section 28, an edge determiner 23A, an image processor 24A, a multiresolution recomposition section 29 and a scan converter 25 which are connected in sequence from the image data generator 20.

The multiresolution decomposition section 28 generates a plurality of decomposed ultrasound image data having different resolutions based on the adjusted ultrasound image data generated in the resolution adjuster 22, and outputs the plurality of decomposed ultrasound image data to the edge determiner 23A. The edge determiner 23A detects edges of the respective interested structures in the ultrasound image based on the plurality of decomposed ultrasound image data and obtains edge information of each of the structures. Edge information of each of the structures thus obtained is outputted to the image processor 24A together with the decomposed ultrasound image data. The image processor 24A performs image processing on each of the decomposed ultrasound image data and outputs a plurality of processed ultrasound image data having different resolutions to the multiresolution recomposition section 29. The multiresolution recomposition section 29 recomposes the plurality of processed ultrasound image data having different resolutions which were inputted from the image processor 24A to generate a single piece of recomposed ultrasound image data and outputs the recomposed ultrasound image data to the scan converter 25. The scan converter 25 performs the display processing such as raster conversion and gradation processing on the recomposed ultrasound image data generated in the multiresolution recomposition section 29 to generate the display image data which can be displayed on the monitor 13 and outputs the display image data to the display controller 12.

In the embodiment 4, even when a single ultrasound image contains a plurality of structures in different sizes, and suitable resolutions for detection of their edges are different among the structures, edge information of each of the structures is obtained by detecting each edge in the plurality of decomposed ultrasound images having different resolutions, and suitable image processing including denoising or edge enhancement is performed on each of the decomposed ultrasound image data based on the corresponding edge information. Hence, it is possible to obtain an ultrasound image in which each of the interested structures has been subjected to suitable image processing and which allows easy observation of each of the structures.

### Embodiment 5

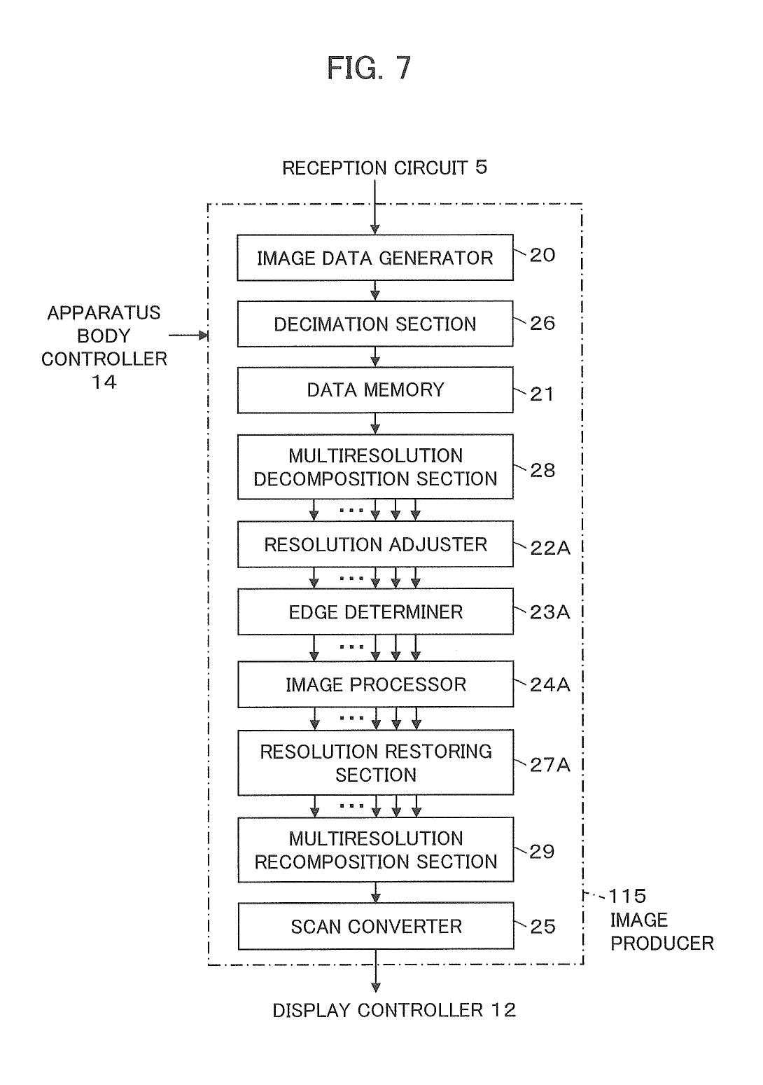

Differently from the above-described embodiment 4, multiresolution decomposition may be performed before adjusting the resolution of the ultrasound image. In an example, as illustrated in FIG. 7, an image producer 115 includes an image data generator 20 connected to the reception circuit 5 of the ultrasound probe 1, and in addition, a decimation section 26, a data memory 21, a multiresolution decomposition section 28, a resolution adjuster 22A, an edge determiner 23A, an image processor 24A, a resolution restoring section 27A, a multiresolution recomposition section 29 and a scan converter 25 which are connected in sequence from the image data generator 20.

The multiresolution decomposition section 28 performs multiresolution decomposition on the downsampled ultrasound image data for one frame outputted from the data memory 21 according to the instruction of the apparatus body controller 14 to generate a plurality of decomposed ultrasound image data having different resolutions and outputs each of the decomposed ultrasound image data to the resolution adjuster 22A. The resolution adjuster 22A adjusts the resolution of each of the decomposed ultrasound image data to generate the adjusted ultrasound image data and outputs the adjusted ultrasound image data to the edge determiner 23A. The edge determiner 23A detects an edge of the interested structure in an ultrasound image corresponding to each of the adjusted ultrasound image data generated in the resolution adjuster 22A to obtain edge information of the structure in each of the adjusted ultrasound image data and outputs the edge information together with the corresponding adjusted ultrasound image data to the image processor 24A. The image processor 24A performs image processing including denoising or edge enhancement on each of the decomposed ultrasound image data based on the edge information of structure therein which was obtained in the edge determiner 23A to generate the processed ultrasound image data and outputs the processed ultrasound image data to the resolution restoring section 27A. The resolution restoring section 27A restores the resolution of each of the processed ultrasound image data such that each of the processed ultrasound image data generated in the image processor 24A has the same resolution as that of the corresponding decomposed ultrasound image data before resolution adjustment in the resolution adjuster 22A and outputs each of the restored image data to the multiresolution recomposition section 29. The multiresolution recomposition section 29 recomposes the plurality of restored ultrasound image data to generate a single piece of recomposed ultrasound image data and outputs the recomposed ultrasound image data to the scan converter 25.

In the embodiment 5, each of the plurality of decomposed ultrasound image data having different resolutions as a result of multiresolution decomposition is independently subjected to resolution adjustment in the resolution adjuster 22A so that edge information of the interested structure can be obtained even in the more appropriate manner than in the embodiment 4. Thus, image processing including denoising or edge enhancement can be appropriately performed based on the suitable edge information, enabling to obtain an ultrasound image in which each of the structures has been subjected to its suitable image processing and which allows easy observation of the respective structures.

### Embodiment 6

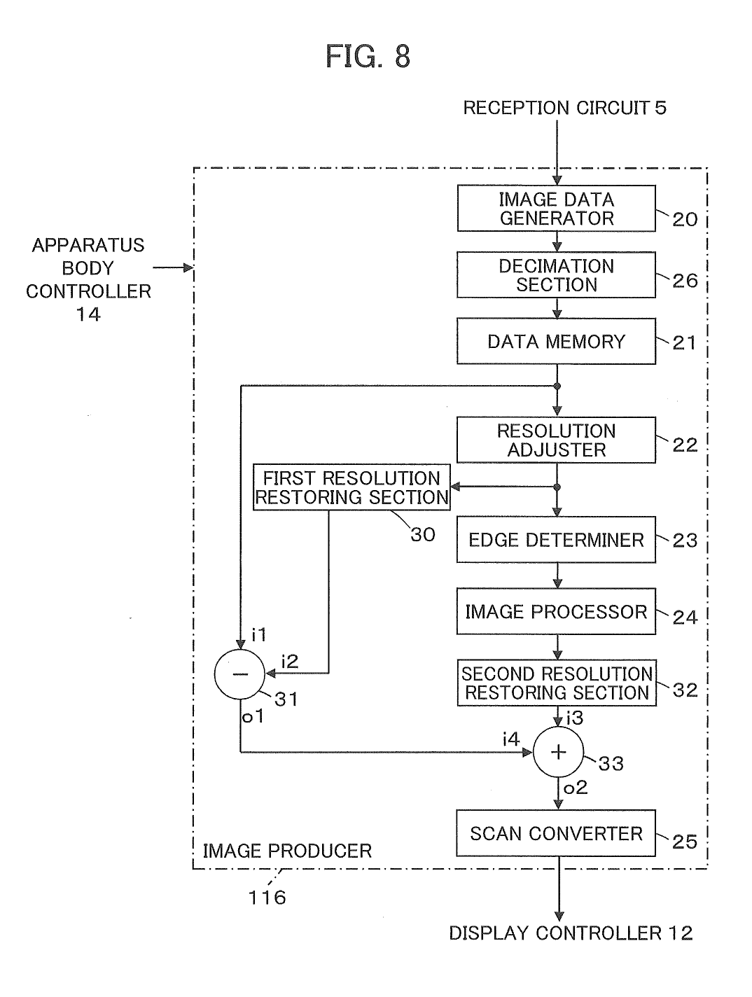

If high frequency components and the like which have been lost due to the resolution adjustment of the ultrasound image data are put back into the ultrasound image data at the end of the series of processes, the information which is lost in the embodiment 2 can be reflected in the ultrasound image. In an example, as illustrated in FIG. 8, an image producer 116 includes an image data generator 20 connected to the reception circuit 5 of the ultrasound probe 1, and in addition, a decimation section 26, a data memory 21, a resolution adjuster 22, an edge determiner 23, and an image processor 24 which are connected in sequence from the image data generator 20. The data memory 21 is connected to the resolution adjuster 22 as described above and also connected to an input terminal i1 of a subtracter 31, while the resolution adjuster 22 is connected to the edge determiner 23 as described above and also connected to a first resolution restoring section 30. The first resolution restoring section 30 is connected to an input terminal i2 of the subtracter 31. In addition, the image processor 24 is connected to a second resolution restoring section 32, and the second resolution restoring section 32 is connected to an input terminal i3 of an adder 33. An output terminal o1 of the subtracter 31 is connected to an input terminal i4 of the adder 33, while an output terminal o2 of the adder 33 is connected to a scan converter 25.

The apparatus body controller 14 outputs the downsampled ultrasound image data for one frame which is stored in the data memory 21 to the resolution adjuster 22 and the input terminal i1 of the subtracter 31, respectively. The resolution adjuster 22 adjusts the resolution of the ultrasound image data inputted from the data memory 21 to generate the adjusted ultrasound image data and outputs the adjusted ultrasound image data to the edge determiner 23 and the first resolution restoring section 30, respectively. The edge determiner 23 and the image processor 24 operate in the same manner as in the embodiment 2. The first resolution restoring section 30 restores the resolution of the adjusted ultrasound image data generated in the resolution adjuster 22 back to the resolution before adjustment in the resolution adjuster 22, to generate the first restored data and outputs the first restored data to the input terminal i2 of the subtracter 31. The subtracter 31 subtracts the first restored data inputted to the input terminal i2 from the downsampled ultrasound image data inputted to the input terminal i1, to generate the subtracted data and outputs the subtracted data to the input terminal i4 of the adder 33 through the output terminal o1 of the subtracter 31. In addition, the second resolution restoring section 32 restores the resolution of the processed ultrasound image data subjected to image processing in the image processor 24 back to the resolution before adjustment in the resolution adjuster 22, to generate the second restored data and outputs the second restored data to the input terminal i3 of the adder 33. Then, the adder 33 adds the subtracted data outputted through the output terminal o1 of the subtracter 31 to the input terminal i4 and the second restored data outputted from the second resolution restoring section 32 to the input terminal i3, to generate the added data and outputs the added data through the output terminal o2 of the adder 33 to the scan converter 25.

In the embodiment 6, since the subtracted data containing the high frequency components which are lost in the embodiment 2 as a result of resolution adjustment is added to the second restored data, it is possible to obtain an ultrasound image maintaining the high frequency components which were to be lost in the resolution adjustment and thus allowing easy observation of the structure.

### Embodiment 7

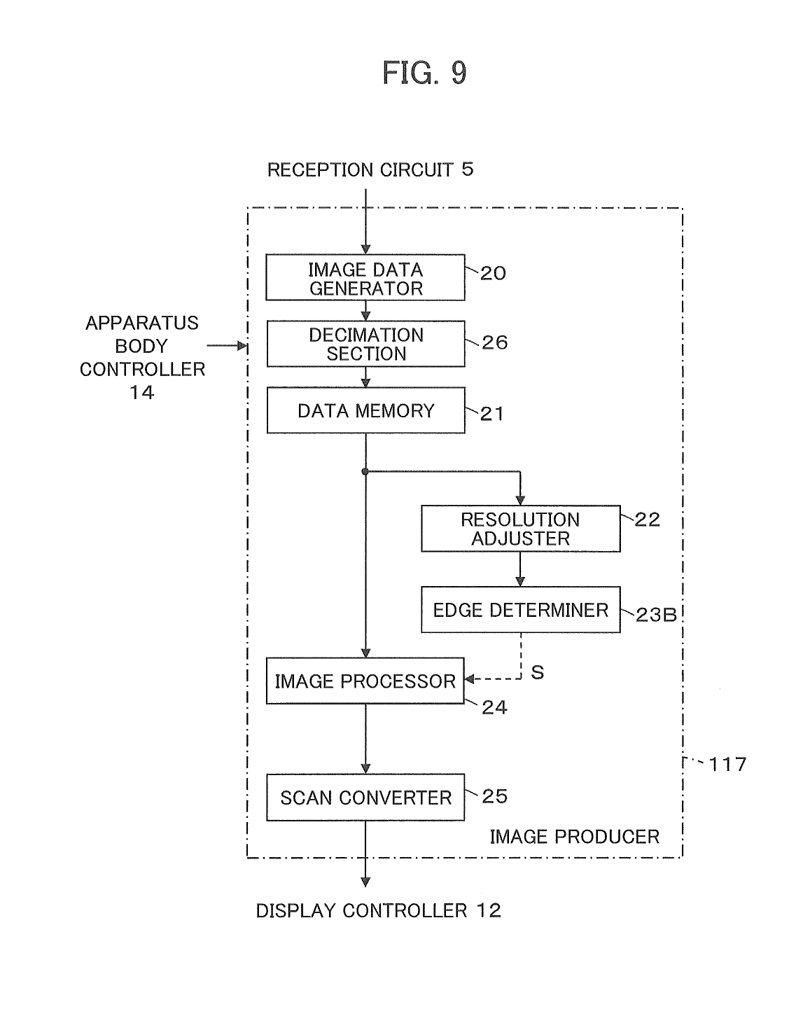

In the above-described embodiment 2, the adjusted ultrasound image data having the adjusted resolution is subjected to image processing including denoising or edge enhancement in the image processor 24 based on the edge information of the structure. However, the ultrasound image data before resolution adjustment in the resolution adjuster 22 may be subjected to image processing including denoising or edge enhancement based on the edge information of the structure. In an example, as illustrated in FIG. 9, an image producer 117 includes an image data generator 20 connected to the reception circuit 5 of the ultrasound probe 1, and in addition, a decimation section 26, a data memory 21, an image processor 24 and a scan converter 25 which are connected in sequence from the image data generator 20. The data memory 21 is connected to the image processor 24 as described above and also connected to a resolution adjuster 22. The resolution adjuster 22 is connected to an edge determiner 23B, and the edge determiner 23B is connected to the image processor 24.

The apparatus body controller 14 outputs the downsampled ultrasound image data for one frame stored in the data memory 21 to the resolution adjuster 22 and the image processor 24, respectively. The resolution adjuster 22 adjusts the resolution of the downsampled ultrasound image data inputted from the data memory 21, to generate the adjusted ultrasound image data and outputs the adjusted ultrasound image data to the edge determiner 23B. The edge determiner 23B obtains the edge information of the structure contained in the corresponding ultrasound image based on the adjusted ultrasound image data generated in the resolution adjuster 22 and outputs the edge information S of the structure to the image processor 24. In the image processor 24, the ultrasound image data outputted from the data memory 21 according to the instruction of the apparatus body controller 14 is subjected to image processing based on the edge information S of the structure outputted from the edge determiner 23 to generate the processed ultrasound image data, and the processed ultrasound image data is then outputted to the scan converter 25.

In the embodiment 7, since the ultrasound image data which has not undergone resolution adjustment is subjected to image processing including denoising or edge enhancement, it is possible to obtain an ultrasound image maintaining information which was to be lost in the resolution adjustment and thus allowing easy observation of the interested structure.

While the ultrasound diagnostic apparatus of the invention has been described above in detail, the invention is by no means limited to the above embodiments, and various improvements and modifications may be made without departing from the scope and spirit of the invention.

What is claimed is:

1. An ultrasound diagnostic apparatus which transmits ultrasound from an ultrasound probe toward a subject and produces an ultrasound image in a diagnostic apparatus body based on reception data obtained, comprising:
an image data generator for generating ultrasound image data based on the reception data,
a resolution adjuster for adjusting a resolution of the ultrasound image data generated in the image data generator to generate adjusted ultrasound image data,
an edge determiner for determining an edge of a structure contained in an ultrasound image based on the adjusted ultrasound image data generated in the resolution adjuster to obtain edge information of the structure, and
an image processor for performing image processing including denoising or edge enhancement on the adjusted ultrasound image data generated in the resolution adjuster or on the ultrasound image data generated in the image data generator based on the edge information obtained in the edge determiner to generate processed ultrasound image data.

2. The ultrasound diagnostic apparatus according to claim 1, further comprising a decimation section for performing decimation on the ultrasound image data to generate downsampled ultrasound image data,
wherein the resolution adjuster adjusts a resolution of the downsampled ultrasound image data generated in the decimation section to generate adjusted ultrasound image data.

3. The ultrasound diagnostic apparatus according to claim 1, wherein the resolution adjuster adjusts the resolution of the ultrasound image data in accordance with a central frequency of the ultrasound probe, a bandwidth of the transmitted ultrasound, a density of scanning lines of the ultrasound probe, filter characteristics of a detection filter for wave detection processing on the reception data, or an imaging technique such as harmonic imaging, to thereby generate the adjusted ultrasound image data.

4. The ultrasound diagnostic apparatus according to claim 1, further comprising a decimation section for performing decimation on the ultrasound image data to generate downsampled ultrasound image data,
wherein the resolution adjuster adjusts a resolution of the downsampled ultrasound image data generated in the decimation section in accordance with a central frequency of the ultrasound probe, a bandwidth of the transmitted ultrasound, a density of scanning lines of the ultrasound probe, filter characteristics of a detection filter for wave detection processing on the reception data, or an imaging technique such as harmonic imaging, to thereby generate adjusted ultrasound image data.

5. The ultrasound diagnostic apparatus according to claim 1, wherein the resolution adjuster adjusts the resolution of the ultrasound image data in proportion to a central frequency of the ultrasound probe to generate the adjusted ultrasound image data.

6. The ultrasound diagnostic apparatus according to claim 1, further comprising a decimation section for performing decimation on the ultrasound image data to generate downsampled ultrasound image data,
wherein the resolution adjuster adjusts a resolution of the downsampled ultrasound image data generated in the decimation section in proportion to a central frequency of the ultrasound probe, to thereby generate adjusted ultrasound image data.

7. The ultrasound diagnostic apparatus according to claim 1, further comprising:
a scan converter for performing coordinate conversion on the ultrasound image data generated in the image data generator for display, and
a resolution restoring section for restoring a resolution of the adjusted ultrasound image data subjected to the image processing in the image processor back to the resolution before adjustment.

8. The ultrasound diagnostic apparatus according to claim 1, further comprising:
a multiresolution decomposition section for performing multiresolution decomposition on the adjusted ultrasound image data generated in the resolution adjuster to decompose the adjusted ultrasound image data into a plurality of decomposed ultrasound image data having different resolutions, and
a multiresolution recomposition section for recomposing the plurality of decomposed ultrasound image data having different resolutions,
wherein the edge determiner determines the edge of the structure contained in the ultrasound image based on the plurality of decomposed ultrasound image data having the different resolutions resulting from decomposition in the multiresolution decomposition section, to thereby obtain the edge information of the structure, and
the image processor performs the image processing on each of the plurality of decomposed ultrasound image data based on the corresponding edge information obtained in the edge determiner to generate a plurality of processed decomposed ultrasound image data having the different resolutions and outputs the plurality of processed decomposed ultrasound image data to the multiresolution recomposition section.

9. The ultrasound diagnostic apparatus according to claim 1, further comprising:
a multiresolution decomposer for performing multiresolution decomposition on the ultrasound image data generated in the image data generator to decompose the ultrasound image data into a plurality of decomposed ultrasound image data having different resolutions,
a resolution restoring section for restoring a resolution of each of the plurality of decomposed ultrasound image data having the different resolutions to generate a plurality of restored decomposed ultrasound image data, and
a multiresolution recomposition section for recomposing the plurality of restored decomposed image data generated in the resolution restoring section,
wherein the resolution adjuster adjusts a resolution of each of the plurality of decomposed ultrasound image data having the different resolutions resulting from multiresolution decomposition in the multiresolution decomposition section to generate a plurality of adjusted decomposed ultrasound image data,
the edge determiner determines the edge of the structure contained in the ultrasound image based on the plurality of adjusted decomposed ultrasound image data generated in the resolution adjuster, to thereby obtain the edge information of the structure, and
the image processor performs the image processing on each of the plurality of adjusted decomposed ultrasound image data based on the corresponding edge information to generate a plurality of processed decomposed ultrasound image data having the different resolutions and outputs the plurality of processed decomposed ultrasound image data to the resolution restoring section.

10. The ultrasound diagnostic apparatus according to claim 1, further comprising:
a first resolution restoring section for restoring a resolution of the adjusted ultrasound image data generated in the resolution adjuster to generate first restored data,
a subtracter for subtracting the first restored data from the ultrasound image data to generate subtracted data,
a second resolution restoring section for restoring a resolution of the processed ultrasound image data generated in the image processor to generate second restored data, and
an adder for adding the subtracted data and the second restored data to generate added data.

11. A method for producing an ultrasound image in which ultrasound is transmitted from an ultrasound probe toward a subject and the ultrasound image is generated based on reception data obtained, comprising the steps of:
generating ultrasound image data based on the reception data,
adjusting a resolution of the generated ultrasound image data to generate adjusted ultrasound image data,
determining an edge of a structure contained in the ultrasound image based on the adjusted ultrasound image data thus generated to obtain edge information of the structure, and
performing image processing including denoising or edge enhancement on the adjusted ultrasound image data or the ultrasound image data based on the edge information thus obtained to generate processed ultrasound image data.
